(19) United States
(12) Patent Application Publication
(10) Pub. No.: US 2003/0181840 A1
(43) Pub. Date: Sep. 25, 2003

(54) Cotton swab attachment means

(76) Inventor: Garry Tsaur, Rowland Heights, CA (US)

Correspondence Address: JOE NIEH, 17800 CASTLETON STREET, SUITE 475, CITY OF INDUSTRY, CA, 91748, US

(21) Appl. No.: 10/103,499

(22) Filed: Mar. 21, 2002

(51) Int. Cl. A61M 35/00

(52) U.S. Cl. 604/1

(57) ABSTRACT

An attachment means is disclosed for reliably and securely attaching cotton, sponge, or other absorbent materials to the ends of cotton swabs tubes. The attachment means comprises of an enlarged contact surface that provides increased friction as well as increased physical resistance to retain the cotton, sponge, or other absorbent material to the ends of the cotton swab tubes. The simple design may be economically incorporated into existing manufacturing process with minimal modification of the manufacturing process.

### BACKGROUND

1. Field of Invention

The present invention relates to an attachment means for securely attaching cotton, sponge, or other absorbent materials to the ends of cotton swabs tubes.

2. Description of Related Art

Cotton swab tubes generally have a plastic tubular housing with cotton, sponge, or other absorbent materials attached to its ends with adhesives. When the cotton swab tubes are used under dry conditions, without contact with any solvents, this attachment means is satisfactory and will hold the cotton, sponge, or other absorbent material securely to the end of the cotton swab tubes. However, if the cotton swab tubes are placed in contact with solvents or any chemical that will dissolve the adhesives used to attach the cotton, sponge, or other absorbent material to the cotton swab tubes the adhesive will not be able to hold the cotton, sponge, or other absorbent materials to the cotton swab tubes.

One design that is utilized to increase the retention of the cotton, sponge, or other absorbent material to the cotton swab tubes when they are placed in contact with solvents is to cut channels on the ends of the cotton swab tubes and attach the cotton, sponge, or other absorbent material on the channeled ends with adhesive. Another design that attempts to minimize the separation of the cotton, sponge, or other absorbent material from the end of the cotton swab tubes is to create a roughened surface at the end of the tubes where the cotton, sponge, or other absorbent materials are attached. However, neither of these designs will hold the cotton, sponge, or other absorbent material securely and consistently when the cotton swab tubes are placed in contact with solvents. Both designs attempt to utilize the limited amount of friction created to retain the cotton, sponge, or other absorbent material when the adhesives are dissolved by the solvent. However, the limited amount of friction cannot reliably retain the cotton, sponge, or other absorbent, material after the adhesives are dissolved by the solvents.

The present invention is a simple design that can be economically adapted to existing manufacturing process to consistently and reliably retain the cotton, sponge, or other absorbent material securely to the ends of the cotton swab tubes even if the adhesives are dissolved by the solvents and even if the adhesives are not used originally to attach the cotton, sponge, or other absorbent material.

### SUMMARY OF THE INVENTION

The present invention relates to an attachment means for reliably and securely attaching cotton, sponge, or other absorbent materials to the ends of cotton swabs tubes. The present invention comprises of an enlarged contact surface that provides increased friction as well as increased physical resistance to retain the cotton, sponge, or other absorbent material to the ends of the cotton swab tubes. The simple design may be economically incorporated into existing manufacturing process with minimal modification of the manufacturing process.

### BRIEF DESCRIPTION OF THE DRAWINGS

FIG. 1 shows the prior art for a cotton swab tube.

FIG. 2 shows the absorbent material detached from the prior art cotton swab tube after the adhesives are dissolved by the solvents.

FIG. 3 shows another prior art for a cotton swab tube.

FIG. 4 shows the preferred embodiment of the cotton swab attachment means.

FIG. 5 shows another embodiment of the cotton swab attachment means.

FIG. 6 shows another embodiment of the cotton swab attachment means.

FIG. 7 shows another embodiment of the cotton swab attachment means.

FIG. 8 shows another embodiment of the cotton swab attachment means.

FIG. 9 shows another embodiment of the cotton swab attachment means.

FIG. 10 shows another embodiment of the cotton swab attachment means.

FIG. 11 shows one of the steps in a process that may be utilized to manufacture the p referred embodiment of the cotton swab attachment means.

FIG. 12 shows another one of the steps in a process that may be utilized to manufacture the preferred embodiment of the cotton swab attachment means.

FIG. 13 shows the result of the manufacturing process of the cotton swab attachment means.

FIG. 14 shows one of the steps in a process that may be utilized to manufacture another one of the embodiment of the cotton swab attachment means.

FIG. 15 shows another one of the steps in a process that may be utilized to manufacture another one of the embodiment of the cotton swab attachment means.

FIG. 16 shows the result of the manufacturing process of the cotton swab attachment means.

FIG. 17 shows one of the steps in a process that may be utilized to manufacture another one of the embodiment of the cotton swab attachment means.

FIG. 18 shows the result of the manufacturing process of the cotton swab attachment means.

FIG. 19 shows one of the steps in a process that may be utilized to manufacture another one of the embodiment of the cotton swab attachment means.

FIG. 20 shows one of the steps in a process that may be utilized to manufacture another one of the embodiment of the cotton swab attachment means.

FIG. 21 shows the result of the manufacturing process of the cotton swab attachment means.

FIG. 22 shows one of the steps in a process that may be utilized to manufacture another one of the embodiment of the cotton swab attachment means.

FIG. 23 shows one of the steps in a process that may be utilized to manufacture another one of the embodiment of the cotton swab attachment means.

FIG. 24 shows the result of the manufacturing process of the cotton swab attachment means.

### DETAILED DESCRIPTION OF THE PREFERRED EMBODIMENT

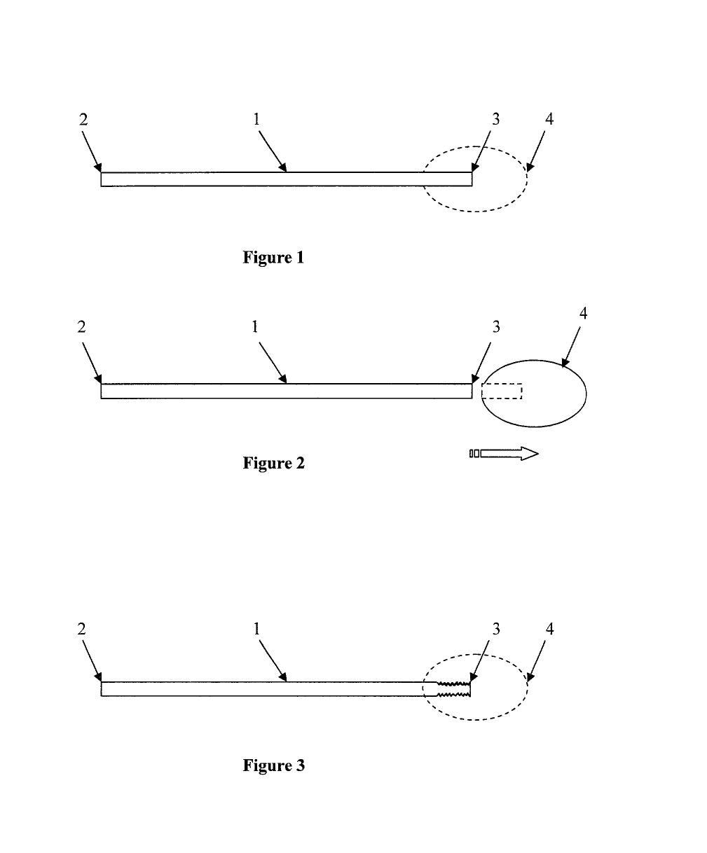

FIG. 1 shows the existing design for a cotton swab tube. The existing design has a tubular housing 1 with absorbent material 4 attached to either or both of its two ends 2, 3 with adhesives. FIG. 2 shows the cotton swab tube with the existing design after it is placed in contact with a solvent that dissolves the adhesives securing the absorbent material 4. Without the adhesives securing the absorbent material 4, the absorbent material 4 will separate from the end 3 of the tubular housing 1 during use. FIG. 3 shows another existing design where channels are cut on the surfaces at the ends 2, 3 to increase the friction with the absorbent material 4. However, this only increases the friction marginally because the force pulling the absorbent material 4 off the end 3 of the tubular housing 1 is tangential to the subsurface grooves.

FIG. 4 shows the preferred embodiment of the cotton swab attachment means. In the preferred embodiment, one or more of the ends 2, 3 with the absorbent material 4 have a larger cross-sectional profile than the remainder of the tubular housing 1. The cross-sectional profile in the preferred embodiment is a rectangle, but the cross-sectional profile may be in any other profiles where the cross-sectional profile is larger than the remainder of the tubular housing 1. This will substantially increase the effective frictional surface between the tubular housing 1 and the absorbent material 4. This will also create a effective physical resistance against the absorbent material 4 separating from the tubular housing 1, whether or not adhesives are utilized.

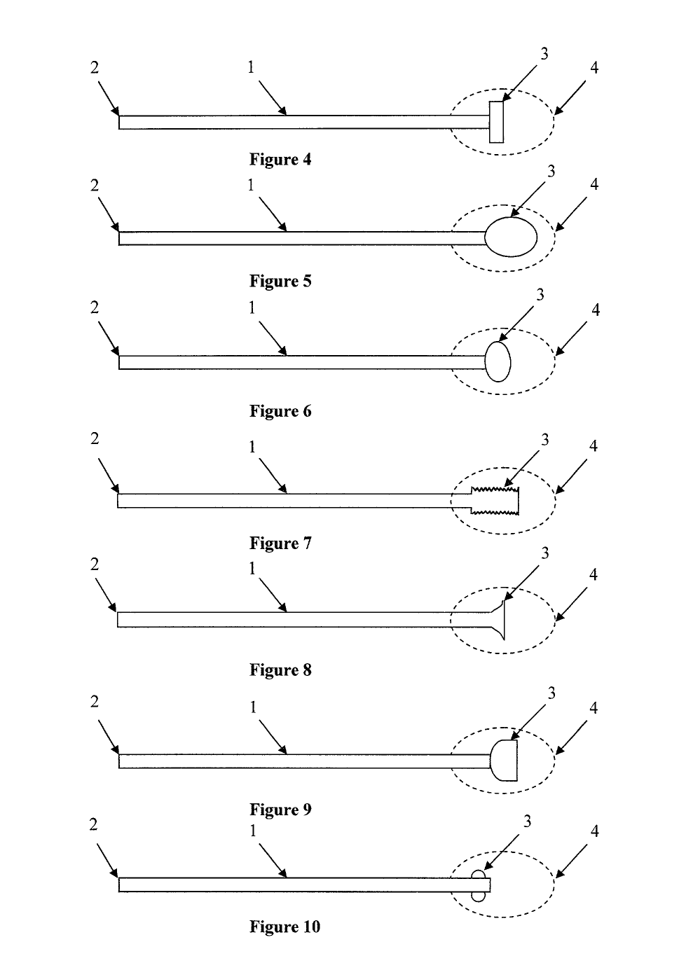

FIG. 5 shows another embodiment of the cotton swab attachment means wherein the cross-sectional profile of one or more of the ends 2, 3 are tangential ellipses. FIG. 6 shows another embodiment of the cotton swab attachment means wherein the cross-sectional profile of one or more of the ends 2, 3 are perpendicular ellipses. FIG. 7 shows another embodiment of the cotton swab attachment means wherein the cross-sectional profile of one or more of the ends 2, 3 is above surface annular rings. FIG. 8 shows another embodiment of the cotton swab attachment means wherein the cross-sectional profile of one or more of the ends 2, 3 is in the shape of a flaring bell. FIG. 9 shows another embodiment of the cotton swab attachment means wherein the cross-sectional profile of one or more of the ends 2, 3 are flattened semicircles. FIG. 10 shows another embodiment of the cotton swab attachment means wherein one or more of the ends 2, 3 have one or more protrusions formed around its circumference.

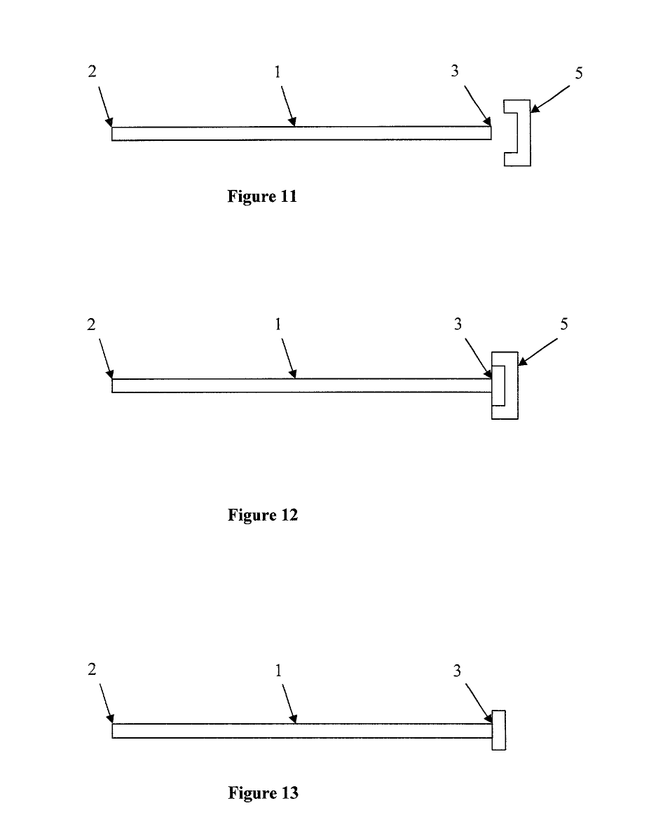

FIG. 11 shows one of the steps in a process that may be utilized to manufacture the preferred embodiment of the cotton swab attachment means. One or more ends 2, 3 of the bare tubular housing 1 is formed into the desired profile by placing it in contact with a heated mold 5. FIG. 12 shows the bare tubular housing 1 with two ends 2, 3 with one of its ends placed in contact with a heated mold 5 with the desired end profile. The heat and pressure from the heated mold 5 will form the ends 2, 3 of the tubular housing 1 into the profile of the heated mold 5 as shown in FIG. 13.

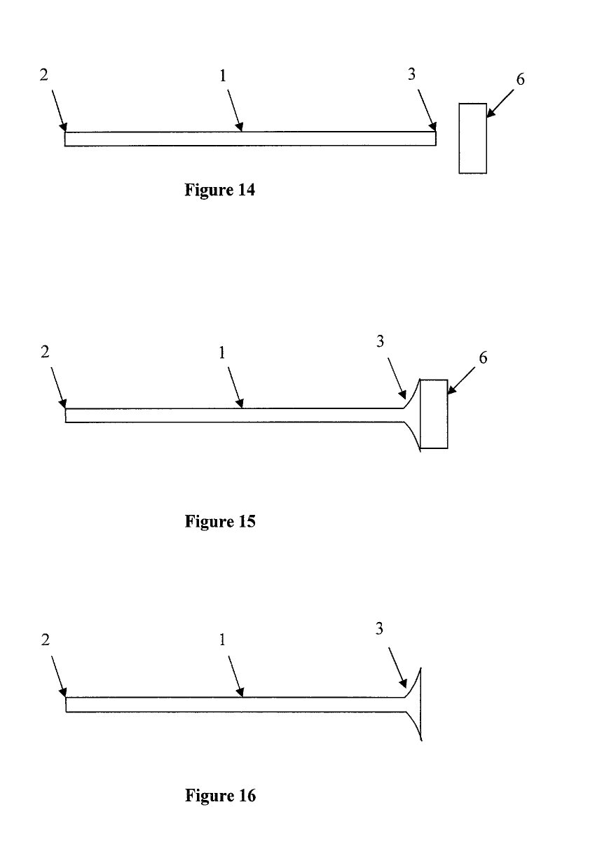

FIG. 14 shows one of the steps in a process that may be utilized to manufacture another one of the embodiment of the cotton swab attachment means wherein the bare tubular housing 1 is placed in contact with a flat heated surface 6. FIG. 15 shows the flaring of the end 3 of the tubular housing 1 due to the pressure and the heat from the flat heated surface 6. The resulting end profile of the flaring bell is shown in FIG. 16.

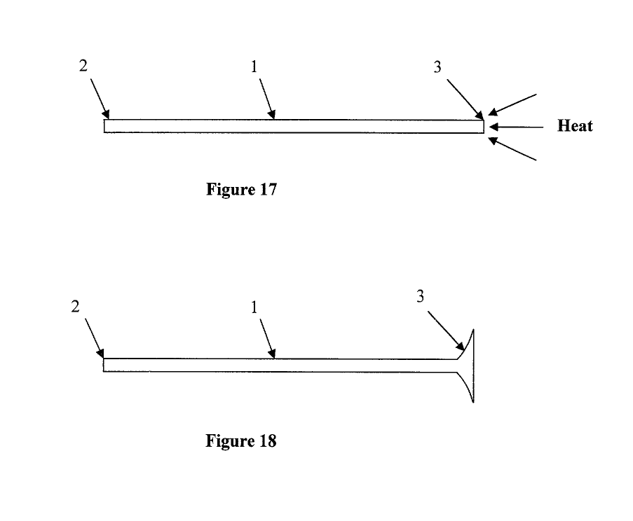

FIG. 17 shows one of the steps in a process that may be utilized to manufacture another one of the embodiment of the cotton swab attachment means. In this process, one or more of the ends 2, 3 are heated with a heat source such as a heat gun causing one or more of the ends 2, 3 on a plastic tubular housing 1 to warp and flare open forming a flaring bell profile as shown in FIG. 18.

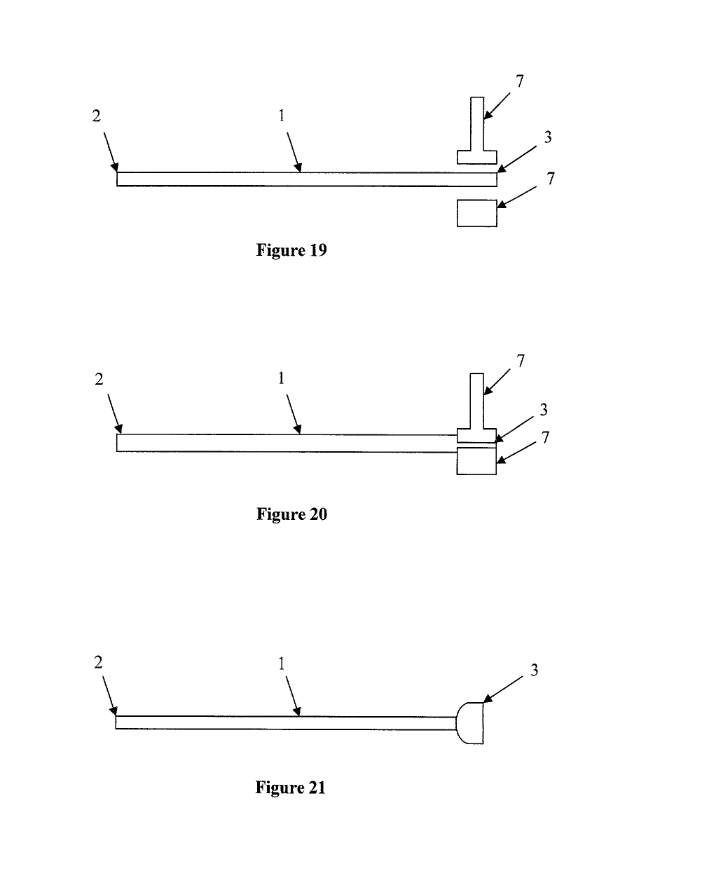

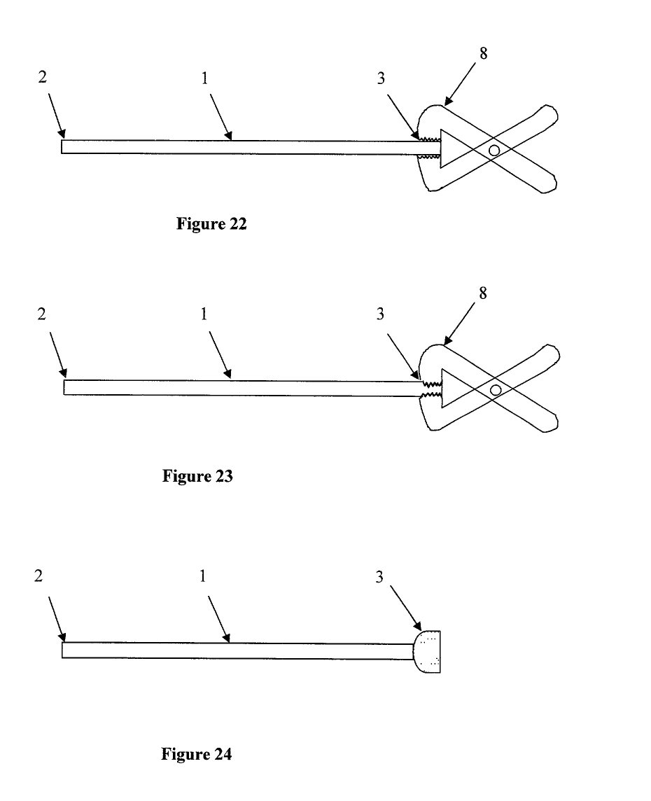

FIG. 19 shows one of the steps in a process that may be utilized to manufacture another one of the embodiment of the cotton swab attachment means. In this process, one or more of the ends 2, 3 are compressed with ultrasound 7 into a flattened shape as shown in FIGS. 20 and 21.

FIG. 22 shows one of the steps in a process that may be utilized to manufacture another one of the embodiment of the cotton swab attachment means. In this process, one or more of the ends 2, 3 are compressed mechanically with a clamping device 8 that will physically flatten one or more of the ends 2, 3 into a flattened profile as shown in FIGS. 23 and 24.

What is claimed is:

1. A cotton swab attachment means comprising:
a tube with two ends wherein one or more of the two ends has a larger cross-sectional profile than the remainder of the tube;
an absorbent material attached to either of the two ends;
wherein the absorbent material is securely and reliably attached to the ends of the tube even if the absorbent material is immersed in a solvent.

2. A cotton swab attachment means as in claim 1, wherein the cross-sectional profile of said end is an ellipse.

3. A cotton swab attachment means as in claim 1, wherein the cross-sectional profile of said end is a rectangle.

4. A cotton swab attachment means as in claim 1, wherein the cross-sectional profile of said end are a series of annular rings.

5. A cotton swab attachment means as in claim 1, wherein the cross-sectional profile of said end is a trapezoid.

6. A cotton swab attachment means as in claim 1, wherein said end is in the profile of a mushroom.

7. A cotton swab attachment means as in claim 1, wherein said end is in the profile of flaring bell.

8. A cotton swab attachment means as in claim 2, 3, 4, 5, 6, or 7, wherein the surfaces on said end is coarse and the coarse surfaces will further increase the friction between the end and the absorbent material and securely retain the absorbent material to said end.
